(19) United States
(12) Patent Application Publication
(10) Pub. No.: US 2025/0364116 A1
(43) Pub. Date: Nov. 27, 2025

(54) SYSTEM AND METHODS FOR MANAGING MEDICAL IMAGING DATA

(71) Applicant: Optum, Inc., Minnetonka, MN (US)

(72) Inventors: Paul Alain Vial, Minnetonka, MN (US); David Dubois, Minnetonka, MN (US); Sara Daneshvar, Minnetonka, MN (US)

(21) Appl. No.: 18/672,358

(22) Filed: May 23, 2024

(51) Int. Cl. G16H 30/20; G06F 40/20

(52) U.S. Cl. CPC G16H 30/20; G06F 40/20

(57) ABSTRACT

Embodiments of the present disclosure provide large language models (LLMs) and large multimodal models (LMMs) for managing imaging data. An example method includes extracting a database schema and a data dictionary associated with a medical imaging database that maintains medical images and non-image data associated with a plurality of clinical studies; extracting object attributes associated with the medical images that are descriptive of the medical images and corresponding studies; and training an LLM to generate search queries from natural language requests based on a training dataset that includes: the database schema, the data dictionary, the object attributes, a prompt template including partial instructions for the LLM, and a plurality of example natural language requests and corresponding expected search queries. The trained LLM can then be used to generate a first search query from a first natural language request.

### BACKGROUND

Medical imaging generally refers to the use of medical imaging technologies to capture images of a subject's body for diagnosing, monitoring, and/or treatment of various conditions and ailments. Medical imaging data is often captured by a medical imaging device (e.g., ultrasound, X-ray, magnetic resonance imaging (MRI), etc.) and communicated to a remote medical imaging database system for processing and/or storage. Commonly, this “medical imaging database system” is a Picture Archiving and Communication System (PACS). Users (e.g., medical professionals, such as radiologists) can remotely access stored medical imaging data from a workstation or other computing device. In many cases, medical imaging data is formatted and exchanged according to the Digital Imaging and Communications in Medicine (DICOM) standard, which defines a data interchange protocol, file format, and structure for medical images and image-related metadata.

In a PACS, a collection of medical images and related information pertaining to a particular patient and examination is referred to as a “study.” A study typically includes a set of medical images from a specific modality such as X-rays, CT scans, MRIs, ultrasounds, and more, as well as associated patient data such as demographic information, examination details, and clinical reports. Within a PACS, medical images from different modalities and time points can be organized and stored together to facilitate efficient retrieval, viewing, and analysis by healthcare professionals. This organization allows radiologists, physicians, and other healthcare providers to access and review all relevant images and information pertaining to a patient's examination in one cohesive package, aiding in diagnosis, treatment planning, and patient care.

### SUMMARY

One implementation of the present disclosure is a system including: at least one processor; and memory having instructions stored thereon that, when executed by the at least one processor, cause the system to: extract a database schema and a data dictionary associated with a medical imaging database; extract object attributes associated with medical images stored in the medical imaging database, wherein the object attributes include data that is descriptive of the medical images and corresponding studies; and train a large language model (LLM) to generate search queries from natural language requests, wherein the LLM is trained on a training dataset that includes: the database schema, the data dictionary, the object attributes, a prompt template including partial instructions for the LLM, and a plurality of example natural language requests and corresponding expected search queries, wherein the trained LLM is used to generate a search query from a natural language request, wherein the search query is executed against the medical imaging database to identify content relevant to the natural language request.

Another implementation of the present disclosure is a computer-implemented method including: extracting, by one or more processors, a database schema and a data dictionary associated with a medical imaging database, wherein the medical imaging database maintains medical images and non-image data associated with a plurality of clinical studies; extracting, by the one or more processors, object attributes associated with the medical images, wherein the object attributes include data that is descriptive of the medical images and corresponding studies; and training, by the one or more processors, a large language model (LLM) to generate search queries from natural language requests, wherein the LLM is trained on a training dataset that includes: the database schema, the data dictionary, the object attributes, a prompt template including partial instructions for the LLM, and a plurality of example natural language requests and corresponding expected search queries, wherein the trained LLM is used to generate a search query from a natural language request, wherein the search query is executed against the medical imaging database to identify content relevant to the natural language request.

Yet another implementation of the present disclosure is a non-transitory computer readable medium having instructions stored thereon that, when executed by at least one processor, cause a computing device to: extract a database schema and a data dictionary associated with a medical imaging database; extract object attributes associated with medical images stored in the medical imaging database, wherein the object attributes include data that is descriptive of the medical images and corresponding studies; and train a large language model (LLM) to generate search queries from natural language requests, wherein the LLM is trained on a training dataset that includes: the database schema, the data dictionary, the object attributes, a prompt template including partial instructions for the LLM, and a plurality of example natural language requests and corresponding expected search queries, wherein the trained LLM is used to generate a search query from a natural language request using the trained LLM, wherein the search query is executed against the medical imaging database to identify content relevant to the natural language request.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 is a diagram comparing process workflows for searching a medical imaging database based on user-provided search requests, according to some implementations.

FIG. 2 is a diagram of process workflows for storing and subsequently searching unstructured data in a medical imaging database, according to some implementations.

FIG. 3 is a block diagram of a medical imaging system, according to some implementations.

FIG. 4 is a flow diagram of a process for training a large language model (LLM) to generate search queries for a medical imaging database based on natural language requests, according to some implementations.

FIG. 5 is a flow diagram of a process for generating an auxiliary database of unstructured data from a medical imaging database using an LLM, according to some implementations.

FIG. 6 is a flow diagram of a process for retrieving content (e.g., studies) from a medical imaging database using one or more trained LLMs, according to some implementations.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a system and methods for managing medical imaging data are shown, according to various implementations. In particular, the disclosed system and methods relate to searching a medical imaging database (e.g., a PACS) to identify content (e.g., studies) that is relevant to a user-provided search request. For example, one use-case of a PACS is to create teaching files, which are used by physicians (e.g., radiologists) to curate and share reference cases. Users may create teaching files by searching a PACS for studies that are relevant to and/or exemplary of a particular medical condition. As noted above, a PACS is perhaps the most common type of medical imaging database; therefore, the system and methods disclosed herein are generally described with respect to a PACS in the interest of brevity. However, it should be appreciated that other types of medical imaging database systems are also contemplated herein and that the disclosed system and methods are not intended to be limited only to use with a PACS.

A PACS commonly maintains structured data and unstructured data, both of which may be useful for identifying relevant studies. In the context of a PACS, structured data generally refers to organized information about patients, medical imaging procedures, and related metadata, as will be appreciated by those in the art. Examples of the types of structured data elements that may be maintained in a PACS may include: patient demographics (e.g., name, date of birth, gender, address, contact details, a unique identifier (such as medical record number or hospital identification number), etc.), exam metadata (e.g., imaging modality, date and time of the procedure, ordering physician identifier, referring department, examination type, etc.), study metadata (e.g., study accession number, study description, study status, etc.), image metadata (e.g., image identifier, acquisition parameters, image orientation, etc.), image storage and retrieval information (e.g., storage location, file format (e.g., DICOM), retrieval timestamps, and access permissions), workflow information (e.g., current stage of image acquisition, interpretation, reporting, and distribution), and audit trail data (e.g., logins, image accesses, modifications, and other security-related events).

Unstructured data in a PACS generally refers to non-standardized or free-text information that does not fit into predefined categories. Examples of the types of unstructured data elements that may be maintained in a PACS may include: clinical reports (e.g., narratives or textual descriptions provided by radiologists or other healthcare professionals interpreting medical images), annotations (e.g., textual or graphical annotations added to medical images by radiologists or technologists to highlight specific findings, anatomical structures, or areas of interest), voice recordings (e.g., dictations or verbal interpretations made by radiologists during image review), diagnostic notes (e.g., observations, hypotheses, or reminders), external documents (e.g., referral letters, medical history forms, consent forms, and prior imaging studies), correspondence (e.g., email communications, messages, or other forms of digital correspondence exchanged between healthcare providers regarding patient care or imaging-related matters), quality control data (e.g., image annotations for training purposes, error logs, and feedback notes), and administrative documents (e.g., records, policies, procedures, and other documents relevant to the operation and management of the PACS).

Typically, a PACS—and/or teaching file creation systems or search tools that are used to search a PACS—provides only a limited number of search fields, for example, in which users can enter search terms to search the PACS. Further, PACSs and/or related search tools are often limited to searching only the structured data maintained in the PACS. In this regard, users are limited to searching just a small number of study attributes (e.g., study date, medical record number, hospital), which is not conducive to efficient searching and building robust teaching files. Many study attributes that could be useful when creating teaching files, for example, as included in the structured data maintained by the PACS, are not searchable, which further limits the results that can be retrieved. Certain teaching file creation systems may enable a user to search the structured data for additional attributes that are most relevant to teaching file creation, but often the user is still limited to a set of search fields that are available on prepared forms (e.g., since the sheer number of attributes that might contain relevant information would be overwhelming to the user).

In addition, unstructured data is generally not searchable using presently available PACS technologies. Certain teaching file creation systems may enable a user to perform a lexical search on unstructured data stored in a PACS (e.g., the teaching file creation system may create an index of radiology reports and store a list of predefined synonyms so that a user can search by keywords or phrases within the reports); however, these lexical search tools are also greatly limited. For example, the lexical search tools that are currently available for use with PACSs are not able to identify sections within a single structured document, for example, such that a user could limit the scope of their search to the “diagnostic impressions” section of a report. Furthermore, these search tools do not capture the semantic properties of words within the context in which they appear (e.g., whether a medical condition was mentioned in regard to a positive finding versus a negative finding). The list of synonyms that may be used by these types of tools is also a potential limitation, in that they must be manually defined, and any omissions will limit users' ability to perform a search.

### Overview

The disclosed system and methods address these and other shortcomings through the use of large language models (LLMs) and/or large multimodal models (LMMs), which enable a user to search both the structured and unstructured data in a PACS to identify studies and/or other content relevant to a request. It should be understood that in various implementations, LMMs can be used in conjunction with or instead of LLMs to execute various functionalities described herein.

Notably, the use of LLMs enables users (e.g., radiologists) to enter search requests using natural (e.g., human) language, as opposed to filling out predefined search fields with limited terms. For example, a user could enter a search request as “I want to create a teaching file to show lung nodules growth” or “show me studies relating to lung nodule growth,” and in turn could be presented with a plurality of studies related to lung nodule growth. As described in greater detail below, an LLM may be trained to generate a search query (e.g., an SQL query) from natural language requests, for example, that considers all the column names in a PACS database. The search query can then be executed in the PACS database, and the resulting studies presented to the user, for example, in a report and/or via a graphical user interface (GUI). In this manner, users are not limited to only a subset of study attributes available on predefined forms (e.g., as mentioned above)—instead, being allowed to utilize any attribute stored in the PACS database that may be relevant to their search without needing detailed knowledge of the PACS database schema. Additional prompt elements or processing may also be applied to rank and sort entries in the result set based on relevance, for example, before presenting to the user.

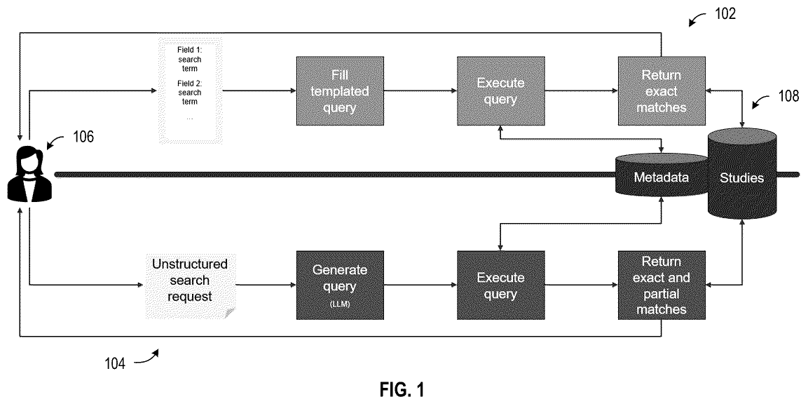

Referring now to FIG. 1, a diagram comparing process workflows for searching a medical imaging database (e.g., a PACS) based on user-provided search requests is shown, according to some implementations. In particular, the top half of the diagram shown in FIG. 1 illustrates a first process workflow 102 representative of how PACS searches are currently performed, while the bottom half of the diagram shown in FIG. 1 illustrates a second process workflow 104 representative of searching a PACS using the system and methods described herein. It should be appreciated that first process workflow 102 is, more specifically, representative of searching the structured data of a PACS since, as discussed above, most presently available PACSs and/or search tools are not capable of searching unstructured data. Accordingly, for comparison, second process workflow 104 is also generally representative of searching the structured data of a PACS.

As shown, first process workflow 102 generally begins with a user 106 (e.g., a radiologist or other medical professional) entering search terms into predefined fields, for example, on an electronic search form. For example, the electronic search form may be in the form of a GUI displayed on a computing device (e.g., a workstation). With reference to the discussion above, the fields of the electronic search form are considered “predefined” in that they are configured to accept only certain types of inputs and/or are associated with a particular study attribute (e.g., study date, medical record number, hospital, etc.). For example, each field may accept only a single term or numerical values as an input, and/or may require a user to select a predefined term/value from a menu. In this regard, existing workflows (e.g., first process workflow 102) for searching a PACS are initially limiting in the structure of a search request. Moreover, formatting a search request in this manner is not intuitive or user-friendly since it requires users to enter specific and limited search parameters.

Once the predefined fields are populated with search parameters (e.g., a term, a number, etc.) and the user initiates a search, the parameters entered into the fields are used to fill a template for generating a query. The query is then executed against a PACS 108 to identify content (e.g., studies) based on the search parameters. As shown, for example, the query may be executed against metadata in the PACS to identify related studies, which are then returned to a user. However, with first process workflow 102, the studies that are identified and returned are typically only exact matches to the search parameters. Therefore, performing searches according to first process workflow 102 can have a significant probability of missing relevant or valuable studies due to the limitations on search parameter entry (e.g., using predefined fields, as discussed above) and/or identifying only those studies that are an exact match to the limited search parameters.

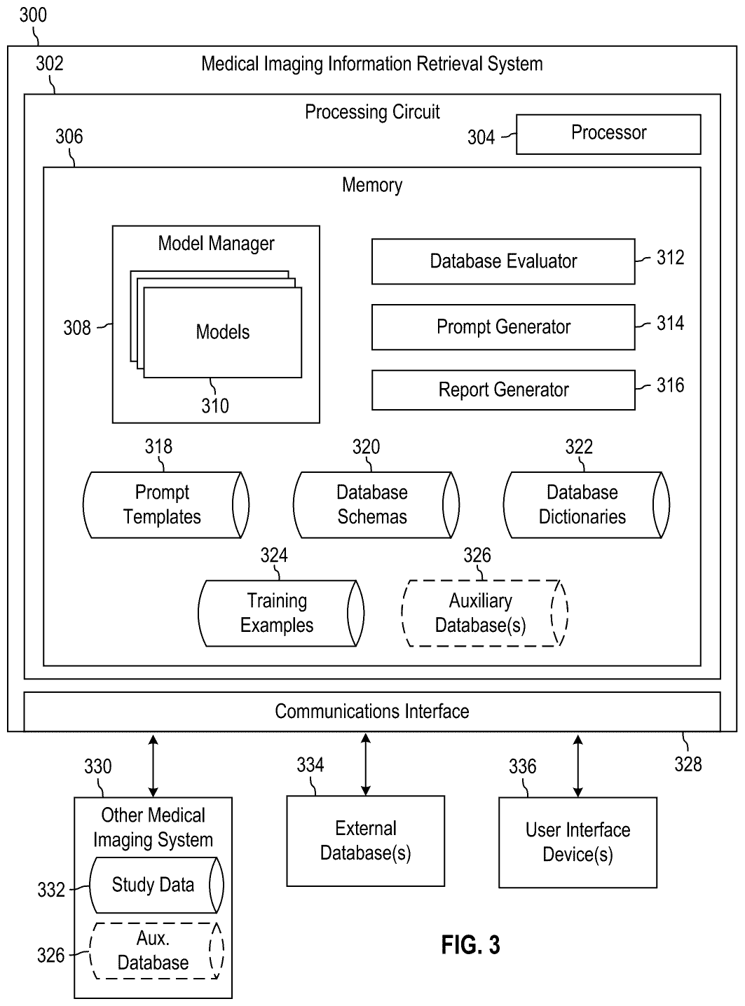

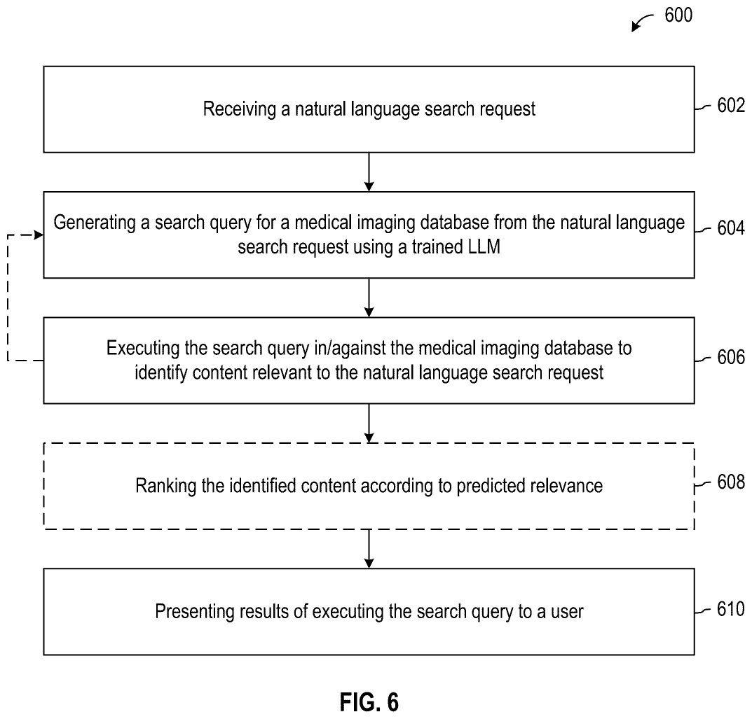

In contrast, the second process workflow 104 begins with user 106 providing an unstructured search request. As described herein, an “unstructured” search request generally refers to a search request that is entered in natural (e.g., human) language. For example, the unstructured search request may be entered as free text into an electronic search form and/or via a GUI. The unstructured search request is then provided as an input to an LLM that has been trained to generate search queries (e.g., an SQL query). As described in greater detail below, for example, the unstructured search request may be inserted into a prompt template which is submitted as a prompt to the trained LLM, which returns a string containing a search query (e.g., an SQL query). The search query generated by the LLM may then be executed against PACS 108 to identify exact and partial matches, which are returned to user 106 (e.g., displayed via a GUI). Additional details relating to second process workflow 104 are discussed below with respect to FIGS. 3 and 6.

Notably, as described herein, an LLM can also be used to search unstructured data in a PACS. For example, rather than returning a string containing a search query, the LLM can be used to search a database containing vectorized representations of the unstructured data in a PACS, for example, to return the most relevant studies. This can enable users to search the unstructured data in a way that captures the syntax and semantics of human language, for example, in both the unstructured data and the user-supplied search prompt. It should further be appreciated that, in this manner, the LLM can identify and take into account different sections within structured data (e.g., the separate sections commonly found within a radiology report separating its content into sections such as history, reason for exam, findings, and impression).

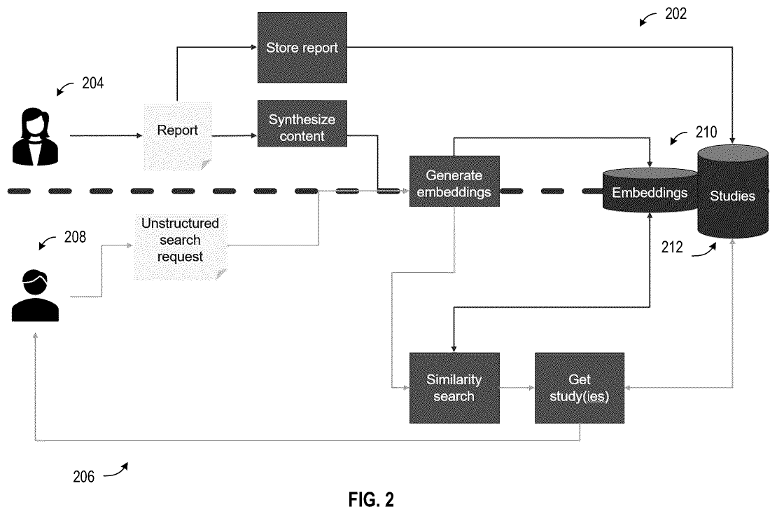

In this regard, FIG. 2 shows a diagram of process workflows for storing and subsequently searching unstructured data in a medical imaging database (e.g., a PACS) using an LLM, according to some implementations. In particular, the top half of the diagram shown in FIG. 2 illustrates a first process workflow 202 initiated by a first user 204 creating or uploading a report (e.g., a text file or other document(s)) to a PACS. As shown, the report is initially stored in a PACS database 212, for example, which can also contain structured and/or unstructured data associated with numerous other studies. Additionally, content may be synthesized from the report. As described herein, synthesizing content from the report may involve converting the textual information in the report into a format that can be integrated with the corresponding medical images and/or studies within PACS database 212. For example, synthesizing content from the report may include extracting keywords, key phrases, and/or other information (e.g., patient demographics, examination details, findings, impressions, recommendations) from the report, and formatting the extracted data into a structured format. This “content synthetization” can be performed using an LLM, LMM, or other model, in some implementations, which is trained to evaluate reports and other text files (e.g., as described below).

In some implementations, the synthesized content can be provided as an input to an embedding model (e.g., a sub-model) which is trained to encode the content (e.g., the report, or elements of the report) as vector embeddings. Accordingly, the embedding model may generate a vector embedding of the synthesized content. The vector embedding may then be stored in a vector database 210 and/or may be used to generate vector database 210 if it is not already created. In other implementations, unstructured data may be provided as a direct input to the embedding model to generate vector embeddings. As described in greater detail below, the vector embeddings contained in vector database 210 may be stored with an identifier or identifiers for the associated study or studies in PACS database 212. In this manner, the vector embeddings in vector database 210 are mapped to corresponding studies in PACS database 212.

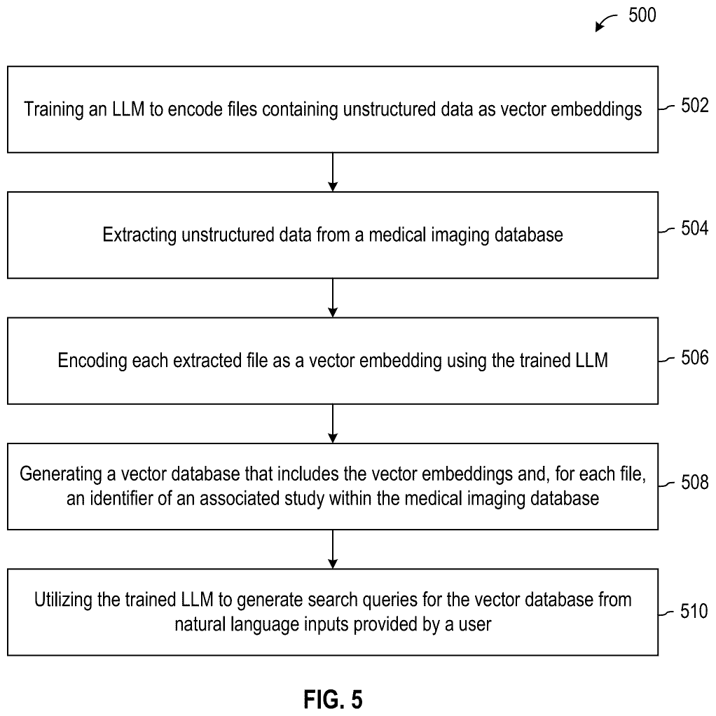

The bottom half of the diagram shown in FIG. 2 illustrates a second process workflow 206 wherein a second user 208 initiates a search (e.g., of PACS database 212) for unstructured data, such as the report created by first user 204. In this case, second user 208 provides an unstructured search request (e.g., a natural language request), for example, via a user interface, which is provided as an input to the LLM (discussed above) trained to generate vector embeddings. The LLM outputs a vector embedding of the unstructured search request which is then used to perform a similarity search to the vector embeddings in vector database 210. In turn, the results of the similarity search can be used to identify unstructured data files (e.g., the report created by first user 204), and related studies in PACS database 212, that are relevant to the unstructured search request. It will be appreciated that, in this regard, the use of LLM embeddings provides a deeper level of semantic understanding and a higher level of accuracy than would be possible using classic embedding types (e.g., word, sentence, bag-of-words, GloVe, etc.). Additional discussion related to first process workflow 202 and second process workflow 206 is provided below with respect to FIGS. 3 and 5.

### Database Management System

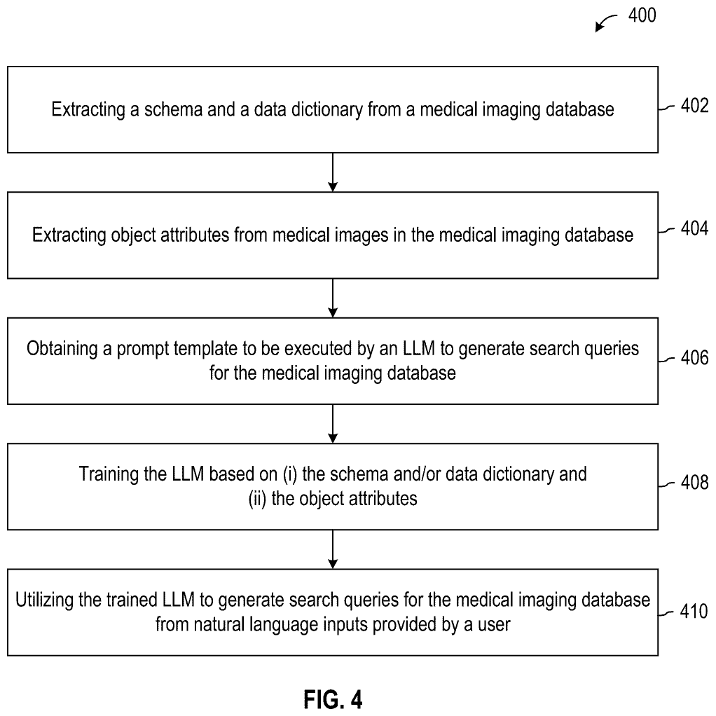

Referring now to FIG. 3, a block diagram of a medical imaging information retrieval system 300 is shown, according to some implementations. As described herein, system 300 may generally be configured to implement the process workflows introduced in FIGS. 1 and 2 and described in greater detail below with respect to FIGS. 4-6. Accordingly, as shown, system 300 may be in communication with another medical imaging system 330, such as, but not limited to PACS, or other medical imaging database to facilitate the management (e.g., storage and retrieval) of medical imaging data. It will be appreciated that system 300 may be external to medical imaging system 330 or may be integrated with (e.g., part of) medical imaging system 330. For clarity, system 300 is generally described herein as a distinct system, for example, with respect to medical imaging system 330, but it should be appreciated that system 300 may be part of medical imaging system 330 in some implementations. In other words, the functionality of system 300 may be implemented by medical imaging system 330 and/or a computing device that hosts medical imaging system 330, or system 300 may be implemented via a separate computing device from medical imaging system 330. As discussed in greater detail below, it should also be appreciated that certain functions and/or components of system 300 can be performed by and/or hosted on a different device, for example, such that system 300 is a distributed system. Therefore, it should be understood that the present disclosure is not intended to be limiting in this regard.

System 300 is shown to include a processing circuit 302 that includes a processor 304 and a memory 306. Processor 304 can be a general-purpose processor, an application-specific integrated circuit (ASIC), one or more field programmable gate arrays (FPGAs), a group of processing components (e.g., a central processing unit (CPU)), or other suitable electronic processing structures. In some implementations, processor 304 is configured to execute program code stored on memory 306 to cause system 300 to perform one or more operations, as described below in greater detail. It will be appreciated that, in implementations where system 300 is part of another computing device (e.g., a PACS, a server, a computer that hosts a PACS, etc.), the components of system 300 may be shared with, or the same as, the host device. For example, if system 300 is implemented via a server, then system 300 may utilize the processing circuit, processor(s), and/or memory of the server to perform the functions described herein.

Memory 306 can include one or more devices (e.g., memory units, memory devices, storage devices, etc.) for storing data and/or computer code for completing and/or facilitating the various processes described in the present disclosure. In some implementations, memory 306 includes tangible (e.g., non-transitory), computer-readable media that stores code or instructions executable by processor 304. Tangible, computer-readable media refers to any physical media that is capable of providing data that causes system 300 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 306 can include random access memory (RAM), read-only memory (ROM), erasable programmable read-only memory (EPROM), electronically erasable programmable read-only memory (EEPROM), hard drive storage, temporary storage, non-volatile memory, flash memory, optical memory, or any other suitable memory for storing software objects and/or computer instructions. Memory 306 can include database components, object code components, script components, or any other type of information structure for supporting the various activities and information structures described in the present disclosure. Memory 306 can be communicably connected to processor 304, such as via processing circuit 302, and can include computer code for executing (e.g., by processor 304) one or more processes described herein.

While shown as individual components, it will be appreciated that processor 304 and/or memory 306 can be implemented using a variety of different types and quantities of processors and memory. For example, processor 304 may represent a single processing device or multiple processing devices. Similarly, memory 306 may represent a single memory device or multiple memory devices. Additionally, in some implementations, system 300 may be implemented within a single computing device (e.g., one server, one housing, etc.). In other implementations, system 300 may be distributed across multiple servers or computers (e.g., that can exist in distributed locations). For example, system 300 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by two or more computers. It should be understood that embodiments of the present disclosure can also include any number of cloud-based components or resources to perform the processes described herein.

Memory 306 is shown to include a model manager 308 that trains and/or otherwise maintains models 310. As described herein, models 310 can include one or more LLMs and/or one or more large multimodal models (LMMs). In particular, models 310 may include at least one LLM that is trained (e.g., by model manager 308) to generate search queries (e.g., an SQL query) for searching medical imaging system 330 based on natural language (e.g., free text) request, as introduced above with respect to FIG. 1. In some implementations, models 310 can include a second LLM trained to encode unstructured data—including natural language (e.g., free text) requests—as vector embeddings, as introduced above with respect to FIG. 2. Alternatively, in some such implementations, models 310 may include just one LLM trained to both generate search queries and encode unstructured data. In some implementations, models 310 includes an LMM that is trained to evaluate medical images (e.g., obtained from medical imaging system 330) to extract additional study attributes. As discussed in greater detail below, for example, the LMM may receive medical images as an input and may output text, for example, indicative of study attributes derived from the medical images.

While shown as part of model manager 308, it should be appreciated that models 310 are not necessarily hosted on memory 306, for example, due to size and/or computational resource limitations. For example, those in the art will appreciate that LLMs and LMMs can be quite sizable (e.g., upwards of 500 GB) and can require significant computing resources for training and/or execution. Accordingly, while described herein with respect to memory 306, it should be appreciated that models 310 may be hosted externally to memory 306 in some implementations. For example, models 310 may be hosted and/or otherwise maintained on a remote server, which model manager 308 accesses to train and/or utilize models 310. Therefore, the specific arrangement of components shown in FIG. 3 is not intended to be limiting.

Model manager 308 generally trains an LLM to generate search queries from natural language requests based on a prompt template and a training data set of example natural language requests and corresponding expected search queries. It should be appreciated that “training” an LLM, in this context, does not necessarily refer to training an LLM from scratch, but rather can be viewed as “fine-tuning” an LLM to a particular use-case. Accordingly, it should be understood that models 310 may include one or more pretrained but generalized LLMs, and that model manager 308 is generally configured to fine-tune models 310 as described herein. In any regard, a prompt template generally includes partial instructions (e.g., part of a prompt) that are provided as an input to the LLM, for example, to cause the LLM to generate an output. For example, in the context of searching medical imaging system 330, the prompt template may be something like “translate this text to an SQL query.” As shown in FIG. 3, prompt templates may be stored in a prompt template database 318, particularly in cases where more than one prompt template is used to train and/or use the LLM. Accordingly, the prompt template may be retrieved from prompt template database 318 and provided as an input to the LLM along with a natural language search request.

For training, the LLM is also provided, as an input, with an example natural language request (e.g., an example of the sort of natural language request that would be provided by a user to initiate a search of medical imaging system 330). In conjunction, the LLM may be provided with an expected output (e.g., an expected search query) for each example natural language request. In this manner, the actual outputs of the LLM can be compared against the expected outputs for training. As those knowledgeable in the art of machine learning will appreciate, parameters (e.g., weights) of the LLM can then be adjusted based on a comparison between the actual output of the LLM and the expected output, for example, to minimize error. As noted above, the example natural language request and corresponding expected search queries may make up a training dataset, which is stored in a training examples database 324. In other words, training examples database 324 may include a plurality of example natural language requests and corresponding expected search queries, which may be manually generated by an expert user.

In addition to the prompt template and the training data set of example natural language requests and corresponding expected search queries, model manager 308 also trains the LLM (e.g., to generate search queries) based on a database schema and a database dictionary of the medical imaging database for which the LLM is being trained to search. For example, in the implementation shown, the LLM is trained based on a database schema and dictionary associated with medical imaging system 330. Generally, a database schema describes the structural representation of the logical and physical layout of a database (e.g., a PACS), which defines the organization, structure, and relationships between data elements and how they are stored. The database schema of medical imaging system 330 can include, for example, a table or tables having columns and rows that define where data is stored. A database dictionary generally includes details about tables, columns, data types, constraints, relationships, indexes, and other database objects as defined by the database schema. In some implementations, the database schema and/or database dictionary are extracted by a database evaluator 312 and/or are stored in corresponding databases—shown as a schema database 320 and a dictionary database 322—as described in greater detail below.

Including a database schema and database dictionary when training and/or executing an LLM can help to reduce or mitigate a phenomenon referred to as “ghosting,” where the LLM generates text that closely resembles or replicates the input it has been given, for example, without demonstrating understanding or originality with respect to the prompt. Ghosting can occur for several reasons, such as overfitting to the prompt, lack of contextual understanding, and inadvertent pattern memorization. To this point, using the database schema and database dictionary of medical imaging system 330 can help to fine-tune the LLM to perform the specific task of generating search queries (e.g., SQL queries) based on natural language inputs.

To further improve results, model manager 308 also trains the LLM based on object attributes extracted from the objects (e.g., medical images) stored in medical imaging system 330. Object attributes refer to data that describes various aspects of the medical images in medical imaging system 330 and related studies. Object attributes can include, for example, patient attributes relating to patient demographics, medical state, and history (e.g., tags from the patient medical module), study attributes that describe the imaging procedure that was performed (e.g., tags from the performed procedure step information), and image attributes that describe the images and their acquisition parameters (e.g., tags from the general series module and modality-specific tags, such as those in the CT Image Module). For example, DICOM defines a wide range of attributes covering various aspects of medical imaging data, including patient demographics, study information, image acquisition parameters, and more. Under the DICOM standard, each attribute is identified by a unique tag consisting of a group number and an element number, which allows for standardized communication and interpretation of medical image data across different systems and vendors. For example, two common DICOM attributes include patient name and image type. Therefore, the “object attributes” described herein can refer to DICOM tags which are extracted from the DICOM headers of the images in medical imaging system 330.

As with the database schema and/or database dictionary described above, in some implementations, object attributes may be extracted or identified by database evaluator 312, as described in greater detail below. Extracted object attributes may be stored in and/or used to generate an auxiliary database 326. In conjunction with the object attributes, model manager 308 can also train LLM using a database schema and a database dictionary associated with the object attributes. For example, where the object attributes are DICOM tags, the database schema and database dictionary are known from the DICOM standard. In some implementations, database evaluator 312 extracts a database schema and database dictionary from auxiliary database 326, for example, after it has been created from the object attributes associated with medical imaging system 330.

To summarize, model manager 308 is configured to train an LLM (e.g., one of models 310) to generate search queries for medical imaging system 330 based on: a database schema and data dictionary extracted from 330, object attributes extracted from medical imaging system 330 and a corresponding database schema and data dictionary, a prompt template, and a training data set of example natural language requests and corresponding expected search queries. Therefore, as an example, the training data used to train the LLM may be formulated as:

```
{
  “in”:
    “Translate this text to an SQL query based on the PACS and
    DICOM database schemas.”
    PACS DB Schema: <stored JSON schema>
    DICOM DB Schema: <stored JSON schema>
    Text: <example free text search request>
  “out”:
    <ideal SQL query based on example free text search request >
}
```

As noted above, model manager 308 may also be configured to train an LLM to encode unstructured data as vector embeddings, such that the unstructured data of medical imaging system 330 can be made searchable. Subsequent to this, in some implementations, a similarity search engine can search a database containing vectorized representations of the unstructured documents and return the most relevant cases. This enables the user to search the unstructured data in a way that captures the syntax and semantics of human language in both the data and the user-supplied search prompt. Additionally, the LLM can identify and take into account different sections within structured documents when searching. As discussed herein, in some implementations, the LLM that is trained to encode unstructured data is the same LLM that is trained to generate search queries (as described above). However, in other implementations, models 310 may include at least two LLMs—one trained to generate search queries and the other to encode unstructured data. In either case, an LLM is fine-tuned (e.g., by model manager 308) to encode unstructured data by training the LLM on a corpus of text files that include the types of documents stored in medical imaging system 330.

In some implementations, models 310 further includes an LMM trained to evaluate medical images (e.g., from medical imaging system 330) and to output text indicative of study attributes derived from the medical images. For example, rather than determine if an image has contrast or not based on rules-based processing of DICOM tags associated with the image, the LMM could evaluate the pixel data of the image to determine if a contrast agent was used, for example, similarly to how a radiologist can tell is a contrast agent was used just by looking at the image. In this regard, models 310 may include an LMM that extracts “derivative study attributes” from pixel data in addition to, or as an alternative to, extracting study attributes solely from the DICOM tags or other metadata associated with the images in medical imaging system 330. Not only can this provide additional information and context relating to a study or image, which may further improve the searchability of medical imaging system 330 using LLMs, as discussed herein, but using an LMM in this manner can also account for potentially incorrect attributes (e.g., DICOM tags).

Database evaluator 312, which is also mentioned above, is generally configured to extract and/or identify data within medical imaging system 330, for example, for the purposes of training models 310. In this regard, database evaluator 312 may be configured to extract a database schema and a database dictionary from medical imaging system 330 which are stored in schema database 320 and dictionary database 322, respectively. Extracting a schema and/or dictionary from a database can generally involve retrieving and documenting the structure of the database, including information about tables, columns, data types, constraints, relationships, and other relevant metadata. It will be appreciated that various techniques for extracting a database schema and/or dictionary are contemplated herein, such as by using SQL queries in the context of medical imaging system 330. In some implementations, the database schema and a database dictionary extracted from medical imaging system 330 are stored in unstructured data format, for example, but not limited to JSON format (e.g., in schema database 320 and dictionary database 322). Specifically, in such implementations, the table and column descriptions from the database dictionary are added to the stored JSON object(s) associated with the database schema. In some implementations, if a database dictionary is not available for medical imaging system 330, database evaluator 312 facilitates the manual creation of one (e.g., by a user).

In some implementations, database evaluator 312 is also configured to extract object attributes from medical imaging system 330. Specifically, database evaluator 312 may evaluate the medical images stored in medical imaging system 330 to extract the object attributes. In implementations where the images in medical imaging system 330 are stored according to the DICOM format, the object attributes (e.g., DICOM tags) can be extracted from the headers of the DICOM images. Database evaluator 312 may store the extracted objected attributes in auxiliary database 326, for example, in unstructured data format. As noted above, the schema and data dictionary for auxiliary database 326 are therefore predefined based on the DICOM standard.

To this point, in some implementations, database evaluator 312 is further configured to execute the LLMs (e.g., models 310) trained by model manager 308 to encode unstructured data (e.g., text files) as vector embeddings. In particular, database evaluator 312 may extract text files that are stored in medical imaging system 330 and may provide the extracted text files as inputs to a trained LLM, which outputs vector embeddings of each text file. In some implementations, database evaluator 312 also extracts text data from the medical images stored in medical imaging system 330 and uses the trained LLM to generate vector embeddings of the text data, for example, so that the additional information that the text data from the medical images contains is searchable. The “text data from the medical images” may be text stored in the headers of DICOM images, for example. In this regard, it will be appreciated that, although the DICOM headers are ostensibly structured data, they often contain long text fields. Therefore, it is useful to treat them as text files or documents for this application. The vector embeddings generated using the trained LLM (e.g., of the text files in medical imaging system 330 and the text files extracted from the DICOM headers of the medical images) are stored in a vector database (e.g., auxiliary database 326) with identifiers that map each vector embedding to a study in medical imaging system 330. In this manner, database evaluator 312 may be considered to “generate” or create auxiliary database 326, for example, by generating vector embeddings of the unstructured data in medical imaging system 330.

Memory 306 is shown to further include a prompt generator 314 which executes the LLMs (e.g., models 310) trained by model manager 308 to generate search queries. In particular, prompt generator 314 is configured to receive a natural language search request, format the natural language search request as a prompt, and provide the prompt as an input to an appropriately trained LLM. As discussed above, the LLM will then output a search query (e.g., an SQL query) that can be executed with respect to medical imaging system 330 to identify content (e.g., studies) relevant to the original natural language search request. The natural language search request, in some implementations, is received as a user input to a user interface device 336, as discussed below. For example, a user may type their natural language search request into a field on a GUI. Upon receipt, prompt generator 314 inserts the natural language search request into a prompt template and submits the prompt to the trained LLM. In some implementations, when generating the prompt, prompt generator 314 further includes references to the database schema and/or dictation associated with medical imaging system 330 and/or the object attributes (e.g., DICOM tags). Using the example above, the prompt may therefore be formatted as:

```
{
  “Translate this text to an SQL query based on the PACS
and DICOM database schemas.”
  PACS DB Schema: <stored JSON schema>
  DICOM DB Schema: <stored JSON schema>
  Text: <free text search request>
}
```

in some implementations. The LLM responds to the prompt with a search query, which may be an SQL query in some implementations. Then, the search query that is generated using the trained LLM can be submitted to medical imaging system 330 and/or auxiliary database 326 (e.g., a DICOM database management system). In turn, medical imaging system 330 and/or auxiliary database 326 execute the search query to identify content (e.g., studies, medical images, etc.) that is relevant to the natural language search request.

In addition, prompt generator 314 may be configured to execute the LLMs (e.g., models 310) trained by model manager 308 to encode unstructured data (e.g., text files) as vector embeddings, in order to search auxiliary database 326. For example, as discussed above, database evaluator 312 may use a trained LLM to generate vector embeddings of the unstructured data in medical imaging system 330. Accordingly, prompt generator 314 may use the same LLM or a different trained LLM to convert a natural language search request (e.g., a user input) to a vector embedding. The generated vector embedding of the natural language search request can then be submitted to auxiliary database 326 (e.g., a vector database) as a query. In some implementations, a similarity search engine or script executes the query, for example, as a similarity search, and returns a result set containing the text files (e.g., documents) that are most relevant to the natural language search request.

Memory 306 is shown to further include a report generator 316 that receives (e.g., from medical imaging system 330 and/or auxiliary database 326) the results of executing the search query and/or vector embedding generated from a natural language search request. As mentioned, the “results” are generally a list or set of studies that are determined to be most relevant to the original search request submitted by a user. In some implementations, report generator 316 is configured to generate a report that lists the results and/or provides a link or other identifier for each study. In some such implementations, the report can be presented as a GUI (e.g., via user interface device 336). For example, report generator 316 may cause an interactive GUI to be presented to a user which lists the results of the search initiated from their natural language prompt. The GUI may be considered “interactive” because user inputs can be interpreted to manipulate the results. For example, the user may select a link associated with a result to cause a second GUI that shows details of the study (e.g., text data, medical images, etc.) to be displayed. In some implementations, report generator 316 is configured to rank results according to relevancy. For example, the LLM(s) that are used to find the results may also output relevancy scores used to rank the results.

Still referring to FIG. 3, system 300 is shown to further include a communications interface 328 that facilitates communications between system 300 and any external components or devices, including medical imaging system 330. In other words, data is transmitted to external recipients and/or received from external sources via communications interface 328. Communications interface 328 can accordingly be or include a wired or wireless communications interface (e.g., jacks, antennas, transmitters, receivers, transceivers, wire terminals, etc.) for conducting data communications, and/or can be or include any combination of wired and/or wireless communication interfaces. Communications via communications interface 328 may be direct (e.g., local wired or wireless communications) or via a network (e.g., a WAN, the Internet, a cellular network, etc.). For example, communications interface 328 may include one or more Ethernet ports for communicably coupling system 300 to a network (e.g., the Internet). In another example, communications interface 328 can include a Wi-Fi transceiver for communicating via a wireless communications network.

medical imaging system 330, as discussed above, is generally representative of any suitable medical imaging database system. For example, while a PACS is one of the most common types of medical imaging database systems, the present disclosure is not intended to be limiting in this regard. As those in the art will appreciate, a PACS generally maintains a database 332 of study data associated with one or more patients, healthcare providers, imaging modalities, and so on. Generally, each study in database 332 includes one or more medical images and related data describing the patient, provider, imaging modality, etc. In this regard, as mentioned above, one use-case of system 300 is to create teaching files, which are used by physicians (e.g., radiologists) to curate and share reference cases. Users may create teaching files by searching database 332 of medical imaging system 330 for studies that are relevant to and/or exemplary of a particular medical condition using system 300 (e.g., by entering search requests in natural language). It should also be appreciated that, in some implementations, medical imaging system 330 can be configured to store data in addition to the study data of database 332. For example, as shown, system 300 may be configured to establish auxiliary database 326 on medical imaging system 330, for example, to offload storage and/or processing requirements.

Building on this point, it should be appreciated that any of the databases and/or data described herein with respect to system 300 may instead be hosted on or otherwise associated with one or more external databases. An external database is any database that is not directly hosted on memory 306 or otherwise maintained by system 300. Accordingly, system 300 is shown to be in communication with external database(s) 334 which, more generally, may represent any suitable remote computing devices. For example, external database(s) 334 may be hosted on remote servers, in the cloud (e.g., on the Internet), and so on. External database(s) 334 could include, for example, cloud storage or additional PACS databases.

User interface device(s) 336 generally includes any devices or components that facilitate user interaction with system 300. For example, in some implementations, user interface device(s) 336 can include one or more external computers (e.g., desktops, laptops, servers, workstations, smartphones, etc.). In some implementations, user interface device(s) 336 includes at least a display device and a user input device. For example, user interface device(s) 336 may include an LED or LCD display for presenting GUIs to a user (e.g., the report generated by report generator 316) and/or a mouse and keyboard for receiving user inputs. In some such implementations, these sorts of display and/or user input devices can be integrated with external computing devices. In any case, user interface device(s) 336 may facilitate displaying data to user(s) and/or receiving inputs (e.g., search requests) from user(s).

### Structured Data

Referring now to FIG. 4, a flow diagram of a process 400 for training an LLM to generate search queries for a medical imaging database (e.g., a PACS) based on natural language requests is shown, according to some implementations. As described herein, process 400 may be implemented by system 300, as described above, or by another suitable computing device. For example, process 400 could be implemented by a server that hosts a PACS or a PACS database management system. It will be appreciated that certain steps of process 400 may be optional and, in some implementations, process 400 may be implemented using less than all of the steps. It will also be appreciated that the order of steps shown in FIG. 4 is not intended to be limiting.

At step 402, process 400 includes extracting a schema and a corresponding data dictionary (e.g., by database evaluator 312) from a medical imaging database (e.g., medical imaging system 330). As discussed above, a schema generally describes the structural representation of the logical and physical layout of the medical imaging database, which defines the organization, structure, and relationships between data elements and how they are stored. A data dictionary generally includes details about tables, columns, data types, constraints, relationships, indexes, and other database objects as defined by the database schema. It should be appreciated that various techniques for extracting a database schema and dictionary are contemplated herein. For example, in the case of a PACS, such as medical imaging system 330, the database schema and dictionary may be extracted using SQL queries (e.g., since a PACS database is generally a type of relational database).

At step 404, process 400 includes extracting object attributes (e.g., by database evaluator 312) from the medical images in the medical imaging database. As also discussed above, object attributes generally include data that describes a medical image or study, for example, such as patient demographics, study information, image acquisition parameters, and more. In the context of a PACS, medical imaging data is often formatted according to the DICOM standard; therefore, “object attributes” may include DICOM tags associated with each medical image. Specifically, the object attributes contemplated herein can include patient attributes relating to patient demographics, medical state, and history (e.g., tags from the patient medical module), study attributes that describe the imaging procedure that was performed (e.g., tags from the performed procedure step information), and image attributes that describe the images and their acquisition parameters (e.g., tags from the general series module and modality-specific tags, such as those in the CT Image Module).

In some implementations, extracting object attributes can further include extracting and/or generating object attribute derivatives. Object attribute derivatives may include useful attributes that are not explicit in the individual object attributes themselves (e.g., the individual raw DICOM tags associated with the medical image data in the medical imaging database). For example, “contrast value” is one type of object attribute that can be extracted and is typically represented as a Boolean value that indicates whether contrast media was used in the acquisition of an image. Contrast value is often defined manually, for example, by an expert user analyzing a number of different DICOM tags that can indicate the presence of contrast (e.g., SeriesDescription, ImageComments, and several of the tags in the Contrast/Bolus Module). In some implementations, an LMM can be used to generate these so-called “derivate” object attributes. For example, the LMM may be trained and/or fine-tuned to evaluate medical images and to output text indicative of study attributes derived from the medical images. For example, rather than determine if an image has contrast or not based on rules-based processing of DICOM tags associated with the image, the LMM could evaluate the pixel data of the image to determine if a contrast agent was used, for example, similarly to how a radiologist can tell if a contrast agent was used just by looking at the image.

As with the database schema and dictionary associated with the medical imaging database itself, object attributes can be extracted using any suitable method, such as via SQL query or by using a suitable object-relational mapping tool. By way of example, an extract-transform-load process in accordance with the present disclosure can include performing at least some of the following steps in relation to a plurality of images: (1) generate a list of DICOM files using SQL or an object-relational mapping tool (2), use software library to read the files themselves, (3) read the DICOM header (e.g., everything except for the for the image's pixel data) for each image in the PACS, (4) transform/restructure the data (e.g., store each study and corresponding attributes or associate each image with a series or study that it is associated with), and (5) load save the data to the auxiliary DB SQL.

In some implementations, the extracted object attributes are used to generate an auxiliary database, for example, of object attributes. In some such implementations, process 400 can further include a step (not shown) of extracting a database schema and dictionary associated with the auxiliary database. The database schema and dictionary for the auxiliary database may be predefined, for example, based on the DICOM standard. For example, if the auxiliary database contains DICOM objects (e.g., if the extracted object attributes are DICOM tags), then the database schema and dictionary are generally defined by the DICOM standard.

At step 406, process 400 includes obtaining a prompt template for using an LLM to generate search queries. As described above, a prompt template is a partial prompt for the LLM which can be supplemented and/or modified. As those in the art will appreciate, a “prompt”—in the context of LLMs—refers to instructions or a request that is provided to the LLM to generate an output. In some implementations, the prompt template is obtained via a user input. Alternatively, or in addition, the prompt template is predefined and retrieved (e.g., from a database) at step 406. In the context of training an LLM to generate search queries for a medical imaging database, the prompt template may include a partial prompt such as “translate this text to an SQL query based on the PACS and DICOM database schemas.” Therefore, as discussed below, it will be appreciated that the prompt may include reference to the schema(s) and/or dictionaries mentioned above. An example prompt template is provided above, with respect to FIG. 3.

At step 408, process 400 includes training the LLM (e.g., by model manager 308) based on the schema and data dictionary extracted from the medical imaging database and the object attributes. As noted above, in some implementations, the LLM may be pre-trained; however, a pre-trained LLM is usually highly generalized. Accordingly, “training” the LLM, in this context, generally refers to “fine-tuning” the LLM for the purposes of generating search queries for a medical imaging database. In any regard, the LLM is generally trained by submitting the prompt template to the LLM, along with the schema and data dictionary extracted from the medical imaging database and data associated with the extracted object attributes. The prompt template may further be supplemented with an example natural language search request and corresponding expected search query. In this manner, the output of the LLM based on the prompt template, schema and data dictionary, object attribute data, and example natural language search request can be compared to an expected search query in order to adjust the LLM. As noted above, in implementations where the extracted object attributes (e.g., from step 404) are used to generate an auxiliary database, the LLM may further be trained on the schema and data dictionary of the auxiliary database. In other words, the LLM is provided—as an input—with the prompt template, the schema and data dictionary of the medical imaging database to be searched, the schema and data dictionary of the auxiliary database (e.g., based on the object attributes), and example natural language search requests.

At step 410, process 400 includes utilizing the trained LLM (e.g., by prompt generator 314) to generate search queries for the medical imaging database from natural language search requests. In other words, after training or “fine-tuning,” the LLM is used to evaluate subsequent natural language search requests, for example, to generate search queries. As discussed above, natural language search requests may be received via a user input, for example, to system 300. The natural language search request may then be provided as an input to the trained LLM, along with the prompt template, the schema and data dictionary of the medical imaging database to be searched, and the schema and data dictionary of the auxiliary database (or the object attribute data). As an output, the LLM outputs a search query which may be, in the context of a PACS, an SQL query. Then, the search query can be automatically executed in/against the medical imaging database to identify content that is relevant to a user's original natural language search request, such as studies or medical images. Additional details are provided below with respect to FIG. 6.

### Unstructured Data

Referring now to FIG. 5, a flow diagram of a process 500 for generating an auxiliary database of unstructured data from a medical imaging database (e.g., a PACS) using an LLM is shown, according to some implementations. As described herein, process 500 may be implemented by system 300, as described above, or by another suitable computing device. For example, process 500 could be implemented by a server that hosts a PACS or a PACS database management system. It will be appreciated that certain steps of process 500 may be optional and, in some implementations, process 500 may be implemented using less than all of the steps. It will also be appreciated that the order of steps shown in FIG. 5 is not intended to be limiting.

At step 502, process 500 includes training an LLM (e.g., by model manager 308) to encode files containing unstructured data (e.g., text files) as vector embeddings. As discussed above, an LLM is trained or fine-tuned to encode unstructured data files based on a corpus of text documents that include the types of documents stored in a PACS or other medical imaging database (e.g., radiology reports, requisitions, ER/ICU opinions, worksheets, pertinent lab data, etc.). For example, the LLM may include a pre-trained but generalized LLM. In some implementations, the LLM that is trained to encode unstructured data files is different from the LLM trained to generate search queries, as described above with respect to FIG. 4. However, in other implementations, one LLM may be trained to both generate search queries and encode unstructured data files, for example, depending on the prompt that is provided to the LLM.

At step 504, process 500 includes extracting unstructured data (e.g., documents, one or more files, sequences of text, or the like) (e.g., by database evaluator 312) from a medical imaging database (e.g., medical imaging system 330). As described above, any suitable extraction technique may be used, such as by SQL query. In some implementations, this can include extracting text from the headers of the images in the medical imaging database, for example, as text files or documents, so that the additional information contained therein will also be searchable. For example, the DICOM headers of images stored in a PACS can be extracted as documents. In some implementations, the text of extracted documents can use a structured data format (e.g. JSON format) to reflect the structure of the DICOM headers. As mentioned above, although the DICOM headers are ostensibly structured data, they contain many text fields that potentially contain useful information, for example, for searching and/or creating teaching files.

At step 506, process 500 includes encoding each extracted file as a vector embedding using the trained LLM (e.g., the LLM embedding model) or another embedding model. In this regard, each text file is provided as an input to the trained LLM, which outputs a corresponding vector embedding. In some implementations, the input(s) to the LLM include a prompt template, such as “encode this text as a vector embedding.”

At step 508, process 500 includes generating a vector database containing the vector embeddings. In other words, the vector embeddings generated using the LLM, at step 506, are compiled and/or stored in a database. Each vector embedding is stored or otherwise associated with an identifier for a corresponding study. For example, the vector embedding of an image header may be mapped (e.g., within the vector database) to a corresponding study in the medical imaging database.

At step 510, process 500 includes utilizing the trained LLM to generate search queries for the vector database from natural language search requests. In other words, after training or “fine-tuning,” the LLM is used to evaluate subsequent natural language search requests, for example, to generate a vector embedding of each natural language search request. The vector embedding of a natural language search request can then be automatically executed in/against the vector database to identify related vector embeddings and thereby corresponding studies. For example, in some implementations, vector embedding of a natural language search request is executed against the vector database using a similarity search function.

### Searching a Medical Imaging Database

Referring now to FIG. 6, a flow diagram of a process 600 for retrieving content (e.g., studies) from a medical imaging database (e.g., a PACS) using one or more trained LLMs is shown, according to some implementations. As described herein, process 600 may be implemented by system 300, as described above, or by another suitable computing device. For example, process 600 could be implemented by a server that hosts a PACS or a PACS database management system. It will be appreciated that certain steps of process 600 may be optional and, in some implementations, process 600 may be implemented using less than all of the steps. It will also be appreciated that the order of steps shown in FIG. 6 is not intended to be limiting.

At step 602, process 600 includes receiving a natural language search request. As mentioned above, a natural language search request refers to a search request provided by a user using natural (e.g., human) language. For example, a natural language search request for medical imaging system 330 could be something like “show me studies relating to lung nodule growth” or “I want to build a teaching file relating to pancreatic cancer.” With respect to FIG. 3, the natural language search request may be received via a user input to a user interface, such as user interface device(s) 336. Alternatively, the natural language search request may be received from a remote computing device or other device. For example, a user could interact with system 300 via a web interface (e.g., by accessing a website) and may enter the natural language search request via a web-based form.

At step 604, process 600 includes generating a search query for a medical imaging database (e.g., medical imaging system 330) from the natural language request using a trained LLM. In particular, the natural language request may be provided as an input to the trained LLM, for example, along with a prompt template, as described above with respect to FIG. 3. Generally, the LLM is also provided with (e.g., as an input) a schema and data dictionary of the medical imaging database to be searched and/or a schema and data dictionary of an auxiliary database that includes object attributes extracted from the medical imaging database. In turn, the LLM outputs a search query based on the natural language request. As mentioned above, for example, in the case of searching a relational database (e.g., medical imaging system 330), the LLM may output an SQL query.

In some implementations, the natural language request is also encoded as a vector embedding using the trained LLM or a second LLM that has been trained accordingly. In other words, step 604 may include generating two search queries from a single natural language request—one search query (e.g., an SQL query) for searching the medical imaging database and another query (e.g., a vector embedding) for searching a vector database that was generated from the unstructured data of the medical imaging database. In this regard, the natural language request received at step 602 can be used to search both the structured and unstructured data of the medical imaging database (e.g., medical imaging system 330).

At step 606, process 600 includes executing the search query in/against the medical imaging database to identify content relevant to the natural language search request. “Relevant content” generally refers to at least one of medical images, structured data, or unstructured data associated with at least one study in the medical imaging database that is related to the user's original request. In other words, the search query generated by the LLM is used to search the medical imaging database for studies that are related to the natural language search request. The identified content may be collated as a “result set,” which is a list or set of identified relevant studies. When the natural language request is also encoded as a vector embedding, as discussed above, step 606 can include executing the vector embedding of the natural language request in/against a vector database (e.g., containing vector embeddings of unstructured data extracted from the medical imaging database, as discussed above), for example, using a similarity search function, to identify unstructured data and related studies that are relevant to the natural language search request.

As shown, it should be appreciated that steps 604 and 606 may be repeated one or more times, in some implementations, such as to search the medical imaging database and then to search the vector database. For example, steps 604 and 606 may be executed initially to generate a search query for searching the medical imaging database, and then can be repeated to generate a vector embedding of the natural language search request (e.g., a “second” search query) for searching the vector database built from unstructured data. Additionally, or alternatively, in some implementations, steps 604 and 606 can be repeated one or more times to run multiple searches of the medical imaging database, for example, based on variations of an original search query. For example, the trained LLM may be executed more than once, based on the natural language search request, to generate multiple search queries that are each slightly different.

At step 608, process 600 includes optionally ranking the results of executing the search query according to relevance. It will be appreciated that various different techniques for ranking the results (e.g., the identified studies) are contemplated herein. For example, the LLM(s) that are used to identify the results or another model/component may also output relevancy scores, which are in turn used to rank the results according to relevancy. In some implementations, the LLM(s) that are used to identify the results, or a separate LLM that is trained differently, could be used to perform a preliminary analysis of the search results to rank the results according to relevancy. As another example, a relevancy formula or model may be applied to generate a relevancy score, for example, based on keywords or other data points extracted from the results.

At step 610, process 600 includes presenting the results of executing the search query to a user. As mentioned above, the “results” can include a list or set of studies that were deemed (e.g., by executing the search query or queries generated by the LLM(s)) relevant to the user's original search request. For example, the results may include identifiers (e.g., a number assigned to each study, links, etc.) associated with each identified study in the medical imaging database. In some implementations, the results are compiled into a report that lists the corresponding study (e.g., by study identifier) and/or that includes a link to each study in the medical imaging database. In some such implementations, the report can be presented as a GUI, such as an interactive GUI that allows for user interaction. For example, the user may select a link associated with a result to cause a second GUI that shows details of the study (e.g., text data, medical images, etc.) to be displayed.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A system comprising:
at least one processor; and
memory having instructions stored thereon that, when executed by the at least one processor, cause the system to:
extract a database schema and a data dictionary associated with a medical imaging database;
extract object attributes associated with medical images stored in the medical imaging database, wherein the object attributes include data that is descriptive of the medical images and corresponding studies; and
train a large language model (LLM) to generate search queries from natural language requests, wherein the LLM is trained on a training dataset that includes: the database schema, the data dictionary, the object attributes, a prompt template comprising partial instructions for the LLM, and a plurality of example natural language requests and corresponding expected search queries,
wherein the trained LLM is used to generate a search query from a natural language request, wherein the search query is executed against the medical imaging database to identify content relevant to the natural language request.

2. The system of claim 1, wherein the medical imaging database is a picture archiving and communication system (PACS), and wherein the object attributes comprise digital imaging and communications in medicine (DICOM) tags.

3. The system of claim 1, wherein the object attributes are stored in an auxiliary database, wherein the instructions further cause the system to determine a second database schema and a second data dictionary associated with the auxiliary database, and wherein the LLM is trained further using the second database schema and a second data dictionary.

4. The system of claim 1, wherein to generate the search query includes to:
receive the natural language request via a user input; and
provide the natural language request as an input to the trained LLM, wherein the trained LLM outputs the search query.

5. The system of claim 1, wherein the content relevant to the natural language request comprises at least one of medical images, structured data, or unstructured data associated with at least one study in the medical imaging database, wherein the instructions further cause the system to present the content relevant to the natural language request via a graphical user interface (GUI).

6. The system of claim 1, wherein the content relevant to the natural language request is ranked according to relevance by the trained LLM.

7. The system of claim 1, wherein the LLM is a first LLM, wherein the instructions further cause the system to:
train a second LLM to encode documents containing unstructured data as vector embeddings, wherein the second LLM is trained using a second training dataset comprising a plurality of text documents that are representative of unstructured data stored in the medical imaging database,
wherein the trained second LLM is used to generate a second search query for the medical imaging database from the natural language request, wherein the second search query is used in conjunction with the search query to identify the content relevant to the natural language request.

8. The system of claim 7, wherein the instructions further cause the system to:
extract a plurality of unstructured data documents from the medical imaging database;
encode each of the plurality of unstructured data documents as a vector embedding using the trained second LLM;
and
generate a vector database that includes, for each of the plurality of unstructured data documents, the vector embedding and an identifier for an associated study in the medical imaging database, wherein the second search query is executed against the vector database.

9. The system of claim 8, wherein to generate the second search query includes to:
receive the natural language request via a user input; and
provide the natural language request as an input to the trained second LLM, wherein the trained second LLM outputs a vector embedding of the natural language request as the second search query, and wherein the vector embedding of the natural language request is executed again the vector database using a similarity search.

10. A computer-implemented method comprising:
extracting, by one or more processors, a database schema and a data dictionary associated with a medical imaging database, wherein the medical imaging database maintains medical images and non-image data associated with a plurality of clinical studies;
extracting, by the one or more processors, object attributes associated with the medical images, wherein the object attributes include data that is descriptive of the medical images and corresponding studies; and
training, by the one or more processors, a large language model (LLM) to generate search queries from natural language requests, wherein the LLM is trained on a training dataset that includes: the database schema, the data dictionary, the object attributes, a prompt template comprising partial instructions for the LLM, and a plurality of example natural language requests and corresponding expected search queries,
wherein the trained LLM is used to generate a search query from a natural language request, wherein the search query is executed against the medical imaging database to identify content relevant to the natural language request.

11. The computer-implemented method of claim 10, wherein the object attributes comprise at least one of:
patient attributes including demographics, a medical state, and a medical history of a patient associated each of the plurality of clinical studies;
study attributes indicative of an imaging procedure that was used to capture the medical images associated with each of the plurality of clinical studies; or
image attributes that describe each of the medical images and their associated acquisition parameters for each of the plurality of clinical studies.

12. The computer-implemented method of claim 10, wherein the medical imaging database is a picture archiving and communication system (PACS), and wherein the object attributes comprise digital imaging and communications in medicine (DICOM) tags.

13. The computer-implemented method of claim 10, wherein the object attributes are stored in an auxiliary database, the method further comprising:
determining a second database schema and a second data dictionary associated with the auxiliary database, wherein the LLM is trained further using the second database schema and a second data dictionary.

14. The computer-implemented method of claim 10, wherein generating the search query includes:
receiving, by the one or more processors, the natural language request via a user input; and
providing, by the one or more processors, the natural language request as an input to the trained LLM, wherein the trained LLM outputs the search query.

15. The computer implemented method of claim 10, wherein the content relevant to the natural language request comprises at least one of medical images, structured data, or
unstructured data associated with at least one study in the medical imaging database, the computer-implemented method further comprising:
presenting, by the one or more processors, the content relevant to the natural language request via a graphical user interface (GUI).

16. The computer-implemented method of claim 10, wherein the content relevant to the natural language request is ranked according to relevance by the trained LLM.

17. The computer-implemented method of claim 10, wherein the LLM is a first LLM, the method further comprising:
training, by the one or more processors, a second LLM to encode documents containing unstructured data as vector embeddings, wherein the second LLM is trained using a second training dataset comprising a plurality of text documents that are representative of unstructured data stored in the medical imaging database,
wherein the second trained LLM is used to generate a second search query for the medical imaging database from the natural language request using the trained second LLM, wherein the second search query is used in conjunction with the search query to identify the content relevant to the natural language request.

18. The computer-implemented method of claim 17, further comprising:
extracting, by the one or more processors, a plurality of unstructured data documents from the medical imaging database;
synthesizing, by the one or more processors, pixel data for each medical image in a selected series to generate a corresponding synthesized content document;
encoding, by the one or more processors and using a large multimodal model (LMM), each corresponding synthesized content document as a vector embedding using the trained second LLM; and
generating, by the one or more processors, a vector database that includes, for each of the plurality of unstructured data documents, the vector embedding and an identifier for an associated study in the medical imaging database, wherein the second search query is executed against the vector database.

19. The computer-implemented method of claim 18, wherein generating the second search query includes:
receiving, by the one or more processors, the natural language request via a user input; and
providing, by the one or more processors, the natural language request as an input to the trained second LLM, wherein the trained second LLM outputs a vector embedding of the natural language request as the second search query, and wherein the vector embedding of the natural language request is executed again the vector database using a similarity search.

20. A non-transitory computer readable medium having instructions stored thereon that, when executed by at least one processor, cause a computing device to:
extract a database schema and a data dictionary associated with a medical imaging database;
extract object attributes associated with medical images stored in the medical imaging database, wherein the object attributes include data that is descriptive of the medical images and corresponding studies; and
train a large language model (LLM) to generate search queries from natural language requests, wherein the LLM is trained on a training dataset that includes: the database schema, the data dictionary, the object attributes, a prompt template comprising partial instructions for the LLM, and a plurality of example natural language requests and corresponding expected search queries,
wherein the trained LLM is used to generate a search query from a natural language request using the trained LLM, wherein the search query is executed against the medical imaging database to identify content relevant to the natural language request.
